(19) United States
(12) Patent Application Publication
(10) Pub. No.: US 2012/0143070 A1
(43) Pub. Date: Jun. 7, 2012

(54) BLOOD PRESSURE DETECTION METHOD AND AN APPARATUS THEREFOR

(76) Inventor: Yiming Yao, Beijing (CN)

(21) Appl. No.: 13/311,901

(22) Filed: Dec. 6, 2011

Related U.S. Application Data
(63) Continuation-in-part of application No. PCT/CN2010/073216, filed on Jun. 30, 2010.

(30) Foreign Application Priority Data
Jun. 30, 2009 (CN) 200910303866.9

(51) Int. Cl. A61B 5/022; A61B 5/021

(52) U.S. Cl. 600/490; 600/485

(57) ABSTRACT

A blood pressure detection method and the apparatus therefor, in which the method includes the steps of recording a voice describing the patient's state when measuring blood pressure and transmitting blood pressure data and voice data to a network server. The network server transforms the blood pressure data of the patient for a given time period into a blood pressure variation curve based upon a plurality of blood pressure measurements. The blood pressure measurement apparatus preferably includes a blood pressure measurement unit with an air pump, an inflatable cuff and a pressure sensor, and a computer processing unit having a CPU, a memory for data storage, a communication interface and a human-machine interface.

### CROSS-REFERENCE TO RELATED APPLICATION

This is a continuation-in-part of P.C.T. International Application No. PCT/CN2010/073216, filed Jun. 30, 2010.

### BACKGROUND OF THE INVENTION

1. Technical Field of the Invention

The present invention relates to a medical detection method and a related apparatus and, more specifically, to a blood pressure detection method and the apparatus therefor.

2. Description of the Prior Art

Blood pressure is one of the three life parameters of human beings. High blood pressure is one of the most common non-infectious chronic diseases and one of the pathogenic factors of other serious chronic diseases.

High blood pressure is, medically, called the “root of all evil,” because long-term high blood pressure will lead to heart, liver and kidney diseases and other complications. Therefore, effective blood pressure control is the basic method to predict and treat many serious diseases harmful to human health.

However, since high blood pressure pathogenesis is very complex and there are many treatment drugs with strong pertinence, an effective drug often loses its potency after being used for a period, thus making it difficult to control. Therefore, high blood pressure patients must detect the blood pressure variation continuously and change the treatment drugs correspondingly, so as to control their blood pressure within a normal range.

In spite of medical advances, there still exist difficulties in the treatment of high blood pressure by patients on blood pressure detection and control regimens, including:

1. The significance of one blood pressure measurement is minimal, since it is normal that the blood pressure of human beings varies during different time periods. Therefore, only measuring blood pressure at many different time points everyday can reflect whether blood pressure variation range is truly normal or not.

2. A doctor or other medical practitioner cannot know other effective information of the time point when the patient measures blood pressure, such as the time after taking drugs, for example, before or after a meal, somatic symptoms, or whether the patients has timely and properly taken the prescribed drugs, and lifestyle.

3. A doctor or other medical practitioner cannot obtain a blood pressure variation curve of the patient in a convenient manner, so the medical professional cannot judge the effectiveness of the treatment resulting from the drug taken by the patient with a given period of time in an effective manner.

Therefore, how to generate a blood pressure variation curve after measuring blood pressure at many different time points and record the patient state information at the measurement point simultaneously has great significance to the doctor and other medical professionals when diagnosing high blood pressure and the drug treatment effect, and formulating an effective treatment scheme for the patient.

### SUMMARY OF THE INVENTION

Accordingly, it is an object of the present invention to provide an effective and convenient blood pressure detection method for detecting blood pressure data and a blood pressure detection apparatus therefor. The blood pressure detection method includes the following steps:

1. When the blood pressure measurer uses the blood detection device to measure blood pressure, a voice describing the measurer's stated information shall be recorded;

2. Transmit the blood pressure value and corresponding voice data to the server of the network data center;

3. The server of the network data center generates the blood pressure data variation curve of the measurer at many different time points within a given time period automatically. The voice corresponding to the measurement points on the blood pressure data variation curve stored in the server can be played via a network terminal computer of the network access server.

4. The blood data variation curve generated by the server of the network data center can be browsed through network terminal and play the record of each measurement point on the network terminal.

As part of the blood pressure detecting method of the present invention is: the measurer is required to record voice information reflecting the patient's state during measurement including: the time after taking drugs, before or after meal, somatic symptoms, whether any other drugs have been taken and, if so, what those drugs would be, the patient's lifestyle, etc. The blood pressure value and the voice data are transmitted to the network server that will transform the blood pressure data of the measurer into a blood pressure variation curve and can play the corresponding voice recorded by the measurer at the measurement point indicated clearly on the curve.

The blood pressure variation curve and the patient's state during measurement are important to the doctor when diagnosing the high blood pressure and the patient's condition accurately. The method makes the submittal of the blood pressure data and state recorded by the measurer during each measurement to the doctor convenient by means of the network.

The blood pressure measurement device having voice recording and network transmission functions used in the blood pressure detection method of the present invention includes a blood pressure measurement unit with an air pump, an inflatable cuff and a pressure sensor, wherein the pressure sensor is connected with a computer processing unit through an A/D conversion interface.

The computer processing unit includes:

a center process unit CPU, used to conduct computing;

a storage, used to store data;

an A/C conversion interface;

a human-machine interface, used to input and display data; and,

buses, used for data transmission between the components;

The computer unit is also connected with a voice collecting device, used to acquire the voice of the user, and a voice module, used to process the voice signals acquired by the voice collecting device and convert them to digital signal.

The communication interface of the computer unit is connected with a data communication module used to transmit the blood pressure data and voice data to the network server.

The inflatable cuff may be an arm or wrist type inflatable cuff.

The communication interface of the computer unit includes, but is not limited to, a serial/parallel communication interface, a USB interface, a network interface, a wired transmission functional interface and a wireless transmission functional interface.

The human-machine interface includes a keyboard, an indicator and a screen.

The data communication module transmitting blood pressure data and voice data is a modulation module of a wireless communication module and a wired network or a mobile communication cell phone.

The blood pressure detection in the present invention is a portable blood pressure measurement instrument, characterized in that: the instrument is capable of recording the voice of the user when measuring and storing the blood pressure data of the user, wherein the user can describe the state information at the measurement time point by means of the voice. The instrument can transmit the blood pressure data and the voice data recorded to the remote network server by means of wired or wireless communication.

The present invention provides a novel manner for treating, controlling and managing the high blood pressure medical condition that features the highest prevalence rate and the biggest threat to human health through the combination of medical instruments, modern communication technology and network technology. The present invention includes the following advantages:

1. Improving the diagnosis accuracy of high blood pressure disease greatly: The variation of human blood pressure in an entire day is normal, so a blood pressure measurement at one time point cannot reveal a problem. While the blood pressure values measured at many time points, especially many feature points in a day are meaningful, the accumulation of blood pressure variation curves over several days can play a subsidiary role in the determination of the patient's state and medication at measurement time points, and provide sufficient bases for the doctor when he/she judges the high blood pressure type and medication effect, which will improve the doctor's diagnosis accuracy of high blood pressure condition.

2. Easy to use and convenient for patient to collect data: If a traditional blood pressure meter is used to record blood pressure variation curve, the patient is required to record the state at a measurement time point on a piece of paper after each measurement. Even if the patient has recorded the data completely, the patient has to then send the data measured to the doctor before obtaining the diagnosis results and the medication scheme. This inefficient method is troublesome to the patient, and it is therefore difficult to ask the patient to operate strictly abiding by this method in “real world” terms.

By contract, with the present invention, which is easy to operate, the patient only needs to measure several times, record short voice recordings in a day and send them by pressing a key after a given time of period. The patient's easy operation and mastery can guarantee the compliant use by patient and, in this way, achieve the purpose of improved diagnosis accuracy of high blood pressure and lead to a more successful treatment regimen.

3. Improve the medical resource use efficiency: High blood pressure patients are required to take drugs all their life, measure their blood pressure and change their drugs under the guidance of doctors. However, there are many high blood pressure patients, so if they all come to the hospital regularly, this will strain medical resources. Moreover, it will waste a lot of energy of the patients and be, generally speaking, wasteful. The present invention enables patients to transmit much more data, and information that is more relevant, to the doctor or other medical professional without the necessity of coming to the hospital or other medical facility. The doctor can judge the condition and medication effect in the given time period according to the information supplied, and instruct the patient in use of more effective treatment methods and drugs; moreover, the present invention enables doctors to provide diagnosis and treatment services for the patients through a network without regional and distance restrictions, thus constantly expanding the medical resource use region and further greatly improving the medical resource use efficiency.

The apparatus used to detect blood pressure data in the present invention is, preferably, a portable instrument. The detection method includes the step of having the user record and store a voice recording describing the user's state when measuring blood pressure; the instrument transmitting the blood pressure data and voice to the remote data terminal or network server by means of wired or wireless communication method; then, the doctor or other medical professional making a more accurate diagnosis according to the blood pressure data curve measured at numerous times and the voice description of the user's state. The present invention will improve the high blood pressure condition control capacity significantly and provide an important method for monitoring and controlling the blood pressure variation of high blood pressure patients effectively.

Other objects and features of the present invention will become apparent when considered in combination with the accompanying drawing figures, which illustrates a certain preferred embodiment of the present invention. It should, however, be noted that the accompanying drawing figures are intended to illustrate only preferred embodiments of the claimed invention and are not intended as a means for defining the limits and scope of the invention.

### BRIEF DESCRIPTION OF THE DRAWING FIGURES

In the accompanying drawing:

FIG. 1 is the structural, or schematic, diagram of the blood pressure detection apparatus of the present invention;

FIG. 2 is a structural, or schematic, diagram of the computing unit of the blood pressure detection apparatus of the present invention;

FIG. 3 is a structural, or schematic, diagram of a blood pressure detection and diagnosis system of the present invention; and,

FIG. 4 is the block diagram of the workflow of a blood pressure detection and diagnosis system.

### DETAILED DESCRIPTION OF THE PREFERRED EMBODIMENTS AND DRAWING FIGURES

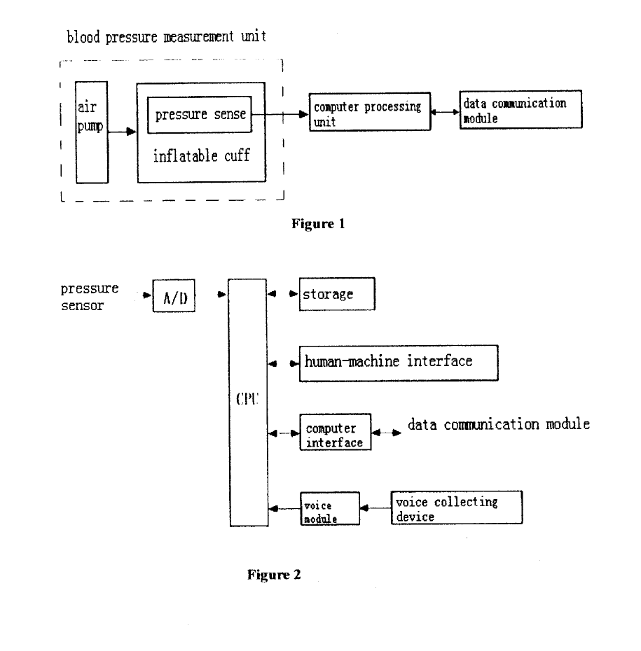

Turning now, in detail, to the accompanying drawing figures, as shown in FIG. 1, the apparatus of the present invention is composed of a blood pressure measurement unit, a computer unit and a data communication module, wherein the composition of the blood pressure measurement unit includes, but is not limited to, a pressure sensor, an air pump and an inflatable cuff, wherein the inflatable cuff may be arm type or wrist type, and the pressure sensor is connected with a computer processing unit through an A/D conversion interface.

As shown in FIG. 2, the computer processing unit includes a center processing unit CPU, a storage, a human-machine interface such as a screen, an indicator and a keyboard, a voice acquisition device such as a voice collecting device, a voice module and a communication interface, wherein: the CPU is the computing and control core of the device, the storage used to store blood pressure data and voice data may be various digital storage, and the communication interface includes a network wired or wireless transmission functional interface and data transmission interfaces between the components inside the device.

A single-chip microcomputer is usually (though not required to be) used in the computer processing unit as a core device, wherein the single-chip microcomputer generally includes a CPU, a storage unit, an A/D interface, an I/O (input/output) interface and a communication interface, etc., and the single-chip microcomputer can extend the storage externally. The keyboard, screen, voice collecting device (e.g, a microphone), LED, data communication module and voice module chip are the external circuit and devices of the single-chip microcomputer. With the higher and higher integration degree of the single-chip microcomputer, many external chips will be integrated inside the single-chip microcomputer.

The data communication module refers to the circuits and devices transmitting the blood pressure data, voice data and other data acquired by the device to the network server or the remote data terminal. The data communication module can be mounted inside the instrument or separated from the instrument. It can transmit data by connecting with the communication interface of the computer processing unit through wired or wireless communication.

A device for detecting blood pressure data in the present invention is, preferably, a portable blood pressure measurement instrument capable of measuring the user's blood pressure anywhere and anytime conveniently. The working principle of the blood pressure measurement unit includes the direct type and indirect type, wherein indirect measurement methods are generally used, and the comparatively more accurate method in indirect methods is the arm-type blood pressure measurement method.

Preferably, the blood pressure data measured every time will be saved in the storage of the device automatically.

An important characteristic of the blood pressure instrument designed in accordance with the present invention is that it can record the user's voice when measuring blood pressure data and, by means of the voice recording, the user can describe the state information at the measurement time point, such as measurement time of day (e.g., in the morning or evening, before or after meal, etc.), body sensation, name and dose of a drug being taken, all of which are very important to the doctor or other medical professional when diagnosing a high blood pressure condition.

The instrument can transmit the blood pressure data and voice data recorded to the remote data terminal or network server through wired or wireless communication, wherein the wired communication methods include wired internet and carrier wave transmitted through telephone line, etc., and the wireless communication methods include, but are not limited to, GPRS data communication method through the GSM network of mobile communication or data transmission method through TD-CDMA or W-CDMA.

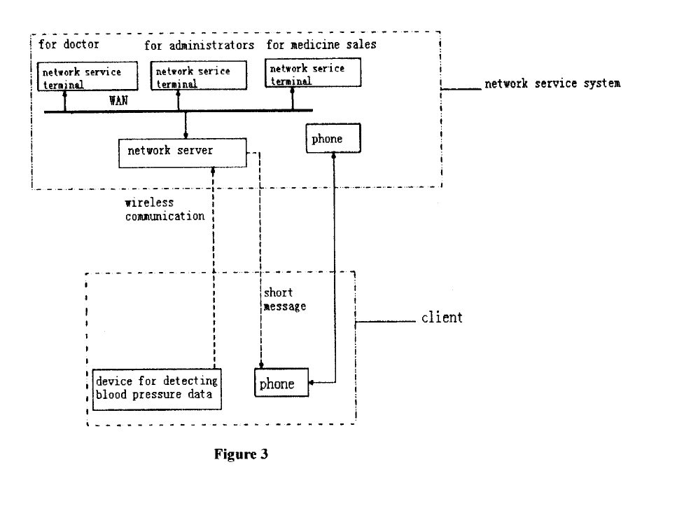

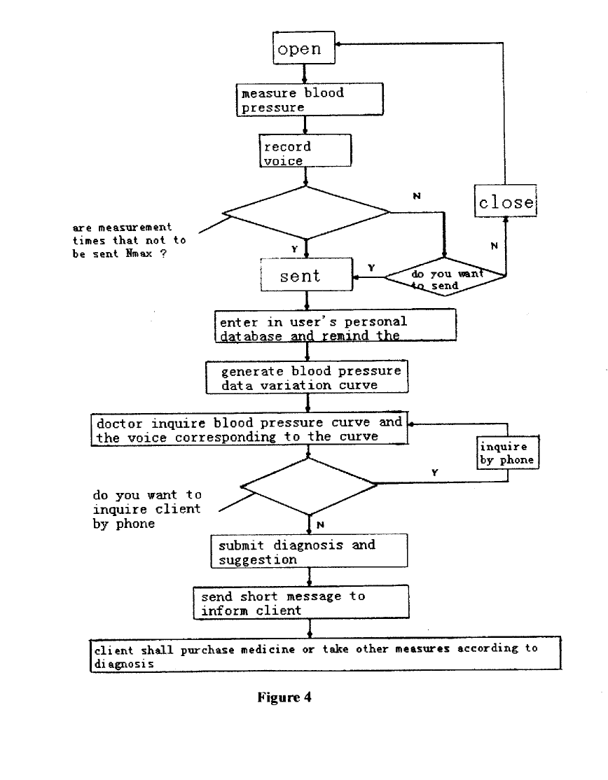

The device is, preferably, a front-data acquisition device based on a network blood pressure detection and diagnosis system. The structural diagram of the blood pressure detection and diagnosis system is as shown in FIG. 3, and includes:

1. A blood pressure detection instrument having voice recording and remote transmission functions in the present invention;

2. Network server;

3. Network terminal; and,

4. The telephone used to enable the customer to communicate with the doctors and other personnel in the service system.

The four parts of devices enumerated above form a networking highly-efficient, high blood pressure disease detection and diagnosis system. The workflow thereof is as shown in FIG. 4.

By using the instrument of the present invention, the patient can measure blood pressure at many time points during the day every day. Generally, the measurement time point shall be chosen at a specific period of time, for example, when waking up in the morning, before or after meal, or the time chosen according to a doctor's instructions; after measurements for certain times (restricted by the storage space, the maximum storage times are Nmax), the patient transmits the data to the network server of the data center or data terminal, the data center and data terminal will generate the blood pressure variation curve in the measurement period and indicate the record at the measurement time points. The doctor can access the patient's record of the measurement time points when observing the blood pressure variation condition, which is equivalent to asking the patient's condition face-to-face. Therefore, the present invention is favorable to the accurate diagnosis and medication scheme of the doctor.

A blood pressure data detection method and the apparatus therefor in accordance with the present invention will be supported a network platform, so the doctor can view the blood pressure curve and state information of the patient and can submit diagnosis and treatment advice through a network terminal; while the patient will access the diagnosis results and treatment advice through the network terminal, wherein the treatment advice may include medication scheme and the customer can purchase high-blood pressure treatment drugs and take other treatment measures according to the doctor's medication advice. The diagnosis and advice of the doctor can, optionally, be sent to the cell phone of the user in a text message or other form of message, which enables the users who do not surf the Internet on a frequent basis to obtain the diagnosis and treatment advice in a timely manner.

Patients' data will preferably be kept in a network database for a long period of time for inquiry of the patients and doctors. Moreover, the blood pressure variation curves of the patients over a long period of time can also be stored. Since there are abundant historical data for the high-blood pressure patients, the doctor is able to track patients over an extended, or long, period of time, resulting in highly efficient treatment of patients by physicians and other medical personnel.

While only several embodiments of the present invention have been shown and described, it will be obvious to those skilled in the art that many modifications may be made to the present invention without departing from the spirit and scope thereof.

What is claimed is:

1. An apparatus for blood pressure data detection, comprising:
a blood pressure measurement unit having an air pump, an inflatable cuff and a pressure sensor;
a computer processing unit to which said pressure sensor is connected via an A/D conversion interface, said computer processing unit comprising a CPU and a memory for storing data, a human-machine interface for inputting and displaying data, and a bus for data transmission;
a voice collecting device connected to said computer processing unit, said voice collecting device for collecting a user's voice and having a voice module for processing signals of the user's voice collected by said voice collecting device and for converting the signals of the user's voice to digital signals;
a data communication module for digitally transmitting data; and,
a communication interface for said computer processing unit connected with said data communication module for digitally transmitting blood pressure data and voice data to a network server.

2. The apparatus for blood pressure data detection according to claim 1, wherein said inflatable cuff of said blood pressure measurement unit is an arm or wrist inflatable cuff.

3. The apparatus for blood pressure data detection according to claim 1, wherein communication interface includes a serial/parallel communication interface, a USB interface, a network interface and a transmission functional interface.

4. The apparatus for blood pressure data detection according to claim 3, wherein said transmission function interface is a wireless transmission functional interface.

5. The apparatus for blood pressure data detection according to claim 3, wherein said transmission function interface is a wired transmission functional interface.

6. The apparatus for blood pressure data detection according to claim 1, wherein said human-machine interface of said computer processing unit includes a keyboard, an indicator and a display screen.

7. The apparatus for blood pressure data detection according to claim 1, wherein said data communication module for digitally transmitting blood pressure data and voice data to a network server is a modulation module of a wireless communication module and a wired network.

8. The apparatus for blood pressure data detection according to claim 1, wherein said data communication module for digitally transmitting blood pressure data and voice data to a network server is a mobile communication cell phone.

9. A method for detecting blood pressure and producing a blood pressure variation, comprising the steps of:
measuring blood pressure of a patient using an apparatus for blood pressure data detection for obtaining blood pressure data for the patient;
audibly recording the blood pressure data for the patient;
digitizing the blood pressure data for the patient that has been audibly recorded;
digitally transmitting the blood pressure data for the patient to a network server of a network data center; and,
generating a blood pressure data variation curve by the network data center based upon blood pressure measurements obtained at a plurality of points in time, the blood pressure data variation curve being accessible to a medical personnel via a network terminal computer.

10. The method for detecting blood pressure and producing a blood pressure variation according to claim 9, wherein said apparatus for blood pressure data detection of said step of measuring blood pressure of patient is performed utilizing a blood pressure measurement unit having an air pump, an inflatable cuff and a blood pressure sensor.

11. The method for detecting blood pressure and producing a blood pressure variation according to claim 9, wherein said step of audibly recording the blood pressure data for the patient and said step of digitizing the blood pressure data for the patient that has been audibly recorded includes:
a computer processing unit to which a blood pressure sensor is connected via an A/D conversion interface, said computer processing unit comprising a CPU and a memory for storing data, a human-machine interface for inputting and displaying data, and a bus for data transmission; and,
a voice collecting device connected to said computer processing unit, said voice collecting device for collecting a user's voice and having a voice module for processing signals of the user's voice collected by said voice collecting device and for converting the signals of the user's voice to digital signals;
a data communication module for digitally transmitting data; and,
a communication interface for said computer processing unit connected with said data communication module for digitally transmitting blood pressure data and voice data to a network server.

12. The method for detecting blood pressure and producing a blood pressure variation according to claim 11, wherein communication interface includes a serial/parallel communication interface, a USB interface, a network interface and a transmission functional interface.

13. The method for detecting blood pressure and producing a blood pressure variation according to claim 12, wherein said transmission function interface is a wireless transmission functional interface.

14. The method for detecting blood pressure and producing a blood pressure variation according to claim 12, wherein said transmission function interface is a wired transmission functional interface.

15. The method for detecting blood pressure and producing a blood pressure variation according to claim 11, wherein said human-machine interface of said computer processing unit includes a keyboard, an indicator and a display screen.

16. The method for detecting blood pressure and producing a blood pressure variation according to claim 11, wherein said data communication module for digitally transmitting blood pressure data and voice data to a network server is a modulation module of a wireless communication module and a wired network.

17. The method for detecting blood pressure and producing a blood pressure variation according to claim 11, wherein said data communication module for digitally transmitting blood pressure data and voice data to a network server is a mobile communication cell phone.
